(19) United States
(12) Patent Application Publication
(10) Pub. No.: US 2024/0350476 A1
(43) Pub. Date: Oct. 24, 2024

(54) Smoking Cessation Aid

(71) Applicant: LUCY GOODS, INC., Las Vegas, NV (US)

(72) Inventors: Ryan Tanakit, Pasadena, CA (US); Samy Hamdouche, Henderson, NV (US); David Renteln, Altadena, CA (US); Tod Miller, Shelbyville, TN (US)

(21) Appl. No.: 18/620,823

(22) Filed: Mar. 28, 2024

Related U.S. Application Data
(60) Provisional application No. 63/455,198, filed on Mar. 28, 2023.

(51) Int. Cl. A61K 31/465; A61K 9/00; A61K 9/70; A61P 25/34

(52) U.S. Cl. CPC A61K 31/465; A61K 9/006; A61K 9/7015; A61P 25/34

(57) ABSTRACT

Disclosed herein are smoking cessation products comprising 6-methylnicotine and methods of treating nicotine addiction using a smoking cessation production comprising 6-methylnicotine.

### CROSS-REFERENCE TO RELATED APPLICATION

The benefit under 35 U.S.C. § 119 (e) of U.S. Provisional Patent Application No. 63/455,198, filed Mar. 28, 2023, is claimed and the entire disclosure thereof is incorporated herein by reference.

### FIELD OF THE DISCLOSURE

The present disclosure relates generally to smoking cessation aids containing 6-methylnicotine. More particularly, the disclosure relates to a pouch containing a 6-methylnicotine salt or complex, wherein the pouch can be used to deliver 6-methylnicotine through the oral mucosa. The disclosure also relates to methods of treating nicotine addiction and methods of treating symptoms of nicotine withdrawal by administering 6-methylnicotine using a smoking cessation product of the disclosure.

### BACKGROUND

Products that contain tobacco leaf, including rolled cigarettes and tobacco products to be packed into the oral cavity, pose health risks to tobacco users due in part to native carcinogens present in tobacco and tar. Nicotine is an addictive chemical that is present in tobacco; tobacco use generally results in quick uptake of nicotine, providing a pleasing effect to the user. It is generally accepted that the addictiveness of nicotine can make it difficult for tobacco users to abstain from using tobacco products, increasing exposure to harmful components present in tobacco and harmful byproducts of tobacco combustion.

Oral smoking cessation products that can provide oral delivery of nicotine to a consumer have been developed. For instance, as described in U.S. Pat. No. 9,161,908B2, use of a sealed paper pouch containing nicotine, to be placed inside a consumer's oral cavity, such as adjacent to the lip, can enable oral delivery of nicotine to a consumer. Oral delivery of nicotine to a consumer can provide the psychoactive sensation provided by nicotine without exposure to harmful byproducts of tobacco combustion. Common modes of consumption of nicotine include traditional cigarettes and electronic cigarettes that involve delivery of nicotine to the lungs via a vaporized mixture with propylene glycol or glycerin. Regardless of delivery method, products of this nature still contain nicotine, and thus may sustain nicotine addiction, and many people who attempt to quit tobacco use through nicotine replacement therapy ultimately relapse to traditional tobacco use.

Producing nicotine for electronic cigarettes and nicotine pouches generally involves extracting the nicotine from tobacco leaf. This extraction can be very energy-intensive, including the use of land and water resources to harvest crops and large amounts of low-cost labor, and also requiring favorable weather conditions. Nicotine can be produced synthetically, but the syntheses are generally expensive, and few manufacturers can produce synthetic nicotine at large scale.

### SUMMARY

To address issues with the use and production of nicotine for smoking cessation products and to address the risks associated with nicotine consumption as part of smoking cessation efforts, research has been conducted to identify alkaloids that can generate effects similar to those generated by nicotine consumption, to help consumers abstain from tobacco and nicotine use by decreasing nicotine withdrawal symptoms that can be experienced during attempts to refrain from tobacco use. Suitable alkaloids can be absorbed through the oral cavity, so that the active ingredient can be presented in similar form factors as synthetic or tobacco-derived nicotine pouches. 6-methylnicotine, in its racemic form, purified to (S)-6-methylnicotine, or purified to (R)-6-methylnicotine, has been found to be a suitable substitute for (S)-nicotine and (R/S)-nicotine. Notably, 6-methylnicotine can be absorbed through the gum line in similar form factors while remaining palatable enough for consumption.

Disclosed herein are smoking cessation products containing 6-methylnicotine and methods of treating nicotine withdrawal symptoms by administering a smoking cessation product containing 6-methylnicotine.

One aspect of the disclosure provides an oral pouch comprising a saliva-permeable non-woven fabric and a composition contained within the oral pouch, the composition comprising a 6-methylnicotine salt or complex and a filler.

Another aspect of the disclosure provides an oral dissolvable film comprising a film-forming saliva-soluble polymer, 6-methylnicotine, and a plasticizer.

Another aspect of the disclosure provides a chewing gum comprising 6-methylnicotine.

Another aspect of the disclosure provides an oral spray or a nasal spray comprising 6-methylnicotine.

Another aspect of the disclosure provides a method of treating a subject for addiction to nicotine, comprising orally administering to a subject an effective amount of 6-methylnicotine to reduce or alleviate nicotine withdrawal symptoms by administering to the subject a smoking cessation product of the disclosure such that the product is placed against the oral mucosa of the subject, wherein upon contact with the oral mucosa, saliva permeates the smoking cessation product and dissolves the composition for absorption of the 6-methylnicotine through the oral mucosa.

For the compositions and methods described herein, optional features, including but not limited to components and compositional ranges thereof, are contemplated to be selected from the various aspects, embodiments, claims, and examples provided herein.

Further aspects and advantages will be apparent to those of ordinary skill in the art from a review of the following detailed description. While the products and methods of the disclosure are susceptible of embodiments in various forms, the description hereafter includes specific embodiments with the understanding that the disclosure is illustrative and is not intended to limit the invention to the specific embodiments described herein.

The smoking cessation products and related methods of treatment are contemplated to include embodiments including any combination of one or more of the additional optional elements, features, and steps further described below, unless stated otherwise.

### BRIEF DESCRIPTION OF THE DRAWINGS

FIGS. 1A-1C show schematic representations of an oral pouch, an oral pouch containing a composition having multiple capsules, and an oral pouch containing a composition having a single capsule, respectively.

FIGS. 2A-2C show schematic representations of side views of an oral pouch, an oral pouch containing a composition having multiple capsules, and an oral pouch containing a composition having a single capsule, respectively.

FIGS. 3A-3C show schematic representations of bottom views of an oral pouch, an oral pouch containing a composition having multiple capsules, and an oral pouch containing a composition having a single capsule, respectively.

FIGS. 4A-4C show schematic representations of top views of an oral pouch, an oral pouch containing a composition having multiple capsules, and an oral pouch containing a composition having a single capsule, respectively.

FIGS. 5A-5C show schematic representations of bottom views of an oral pouch, an oral pouch containing a composition having multiple capsules, and an oral pouch containing a composition having a single capsule, respectively.

### DETAILED DESCRIPTION

A composition for use in a smoking cessation product in accordance with the disclosure can include 6-methylnicotine. a salt thereof, or a complex thereof. The composition can be provided in a variety of forms depending on the administration method for the smoking cessation product. For example, the composition can be provided in an oral administration form, by which an interaction of saliva with the composition releases 6-methylnicotine from the composition for absorption through the oral mucosa.

One aspect of the disclosure provides a smoking cessation product for oral delivery, comprising an oral pouch comprising a saliva-permeable non-woven fabric and a composition contained within the oral pouch, the composition comprising a 6-methylnicotine salt or complex and a filler.

Another aspect of the disclosure provides an oral dissolvable film for delivering 6-methylnicotine to the oral mucosa, comprising a film-forming saliva-soluble polymer and 6-methylnicotine or a 6-methylnicotine salt.

Another aspect of the disclosure provides a method of treating a subject for addiction to nicotine, comprising orally administering to a subject an effective amount of 6-methylnicotine to reduce or eliminate nicotine withdrawal symptoms by administering to the subject a smoking cessation product of the disclosure to be disposed against the oral mucosa of the subject, wherein upon contact with the oral mucosa, saliva permeates the smoking cessation product and dissolves the composition for absorption of the 6-methylnicotine through the oral mucosa.

All ranges set forth herein include all possible subsets of ranges and any combinations of such subset ranges. By default, ranges are inclusive of the stated endpoints, unless stated otherwise. Where a range of values is provided, it is understood that each intervening value between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also contemplated to be part of the disclosure.

The dimensions and values disclosed herein are not to be understood as being strictly limited to the exact numerical values recited. Instead, unless otherwise specified, each such dimension is intended to include both the recited value and a functionally equivalent range surrounding that value. For example, a dimension disclosed as “15 mm” is intended to include “about 15 mm,” and “about 15 mm” can include a range of from 14.5 mm to 15.4 mm, e.g., by numerical rounding.

As used herein and unless specified otherwise, the terms “wt. %” and “wt %” are intended to refer to the amount of the identified element of a composition in “dry” (non-water) parts by weight of the composition.

The smoking cessation products and related methods of treatment and use are contemplated to include embodiments including any combination of one or more of the elements, features, and steps further described below (including those shown in the Examples and Figures), unless stated otherwise.

### 6-Methylnicotine

Nicotine is an addictive chemical that is present in tobacco. Smokers can find it difficult to abstain from tobacco use in part because of this nicotine content, leading to prolonged exposure to harmful materials present in tobacco and generated from tobacco consumption.

Nicotine-containing products, such as nicotine gums, patches, or oral pouches, can be used as part of nicotine replacement therapy (NRT) by consumers attempting to stop using tobacco. Such products can deliver a controlled dose of nicotine to a consumer to provide the satisfactory effects of nicotine without smoking or chewing tobacco. Nicotine doses can vary by product; for instance, nicotine gums typically contain 2 mg or 4 mg of nicotine, and nicotine pouches typically contain from 2 mg to 15 mg of nicotine. However, nicotine itself can produce side effects, and continued consumption of nicotine leaves open the risk that a consumer may relapse into use of tobacco products.

6-methylnicotine is one of many structural variants of nicotine. It has been advantageously found that delivering 6-methylnicotine by using a smoking cessation product according to the disclosure can help treat nicotine addiction and reduce symptoms of nicotine withdrawal by providing satisfactory physiological effects without consumption of nicotine.

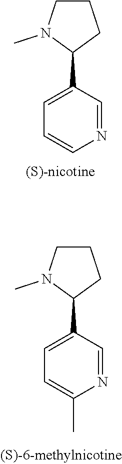

6-methylnicotine can be present the smoking cessation products disclosed herein as 6-methylnicotine (i.e., the “free form” of the molecule), a 6-methylnicotine salt, or a 6-methylnicotine complex.

The 6-methylnicotine salt is not particularly limited. The 6-methylnicotine salt can include, but is not limited to, 6-methylnicotine hydrochloride, 6-methylnicotine dihydrochloride, 6-methylnicotine monotartrate, 6-methylnicotine bitartrate, 6-methylnicotine bitartrate dihydrate, 6-methylnicotine sulphate, 6-methylnicotine malate, 6-methylnicotine benzoate, 6-methylnicotine citrate, 6-methylnicotine levulinate, 6-methylnicotine zinc chloride monohydrate, 6-methylnicotine salicylate, and combinations thereof. Without intending to be bound by theory, it is believed that 6-methylnicotine salts are generally more stable with respect to degradation and oxidation compared to 6-methylnicotine.

The 6-methylnicotine complex can be a complex of 6-methylnicotine with a cyclodextrin (i.e., a 6-methylnicotine cyclodextrin complex) or a complex of 6-methylnicotine with an ion exchange resin (i.e., 6-methylnicotine polacrilex). The 6-methylnicotine cyclodextrin complex can comprise α-cyclodextrin (alpha-cyclodextrin), β-cyclodextrin (beta-cyclodextrin), methyl-β-cyclodextrin (methyl-beta-cyclodextrin), or γ-cyclodextrin (gamma-cyclodextrin). For example, the 6-methylnicotine cyclodextrin complex can be a complex with β-cyclodextrin (beta-cyclodextrin). The ion exchange resin of 6-methylnicotine polacrilex is not particularly limited; strong-acid cation exchange resins and weak-acid cation exchange resins, as will be familiar to those of skill in the art, can be used in the complex. Suitable cation exchange resins include, but are not limited to, Amberlite™ IRP 64, Amberlite™ IRP 69, Amberlite™ IR 120, Dowex™ 50, BIO-REX™ 40, Ionac™ C240, and Duolite™ ES-63. Without intending to be bound by theory, it is believed that 6-methylnicotine complexed with a cyclodextrin or with an ion exchange resin is generally more stable with respect to degradation and oxidation than 6-methylnicotine.

6-methylnicotine can be present in the smoking cessation products of the disclosure as (R)-6-methylnicotine, (S)-6-methylnicotine, or any mixture thereof. In embodiments, the 6-methylnicotine can comprise at least 50% (S)-6-methylnicotine, based on the total amount of 6-methylnicotine, or least 60%, or at least 70%, or at least 80%, or at least 90%, or at least 95%, or 100% (S)-6-methylnicotine.

Smoking cessation products according to the disclosure can deliver an amount of 6-methylnicotine to a consumer sufficient to provide a physiological effect similar to that provided by nicotine consumption. For instance, a smoking cessation product according to the disclosure can contain from about 0.1 mg to about 10 mg, or about 0.5 mg to about 5 mg, or about 1 mg to about 3 mg, of 6-methylnicotine. In embodiments, the amount of 6-methylnicotine that is sufficient to provide a physiological effect is less than the amount of nicotine that would be required to provide the same degree of physiological effect.

### Oral Pouch

The smoking cessation product can be an oral pouch. The pouch includes a composition contained within the pouch, the composition comprising 6-methylnicotine, a 6-methylnicotine salt, a 6-methylnicotine complex, or a combination thereof, and optionally one or more additional components. Preferably the composition comprises a 6-methylnicotine salt or a 6-methylnicotine complex. Use of the pouch can include placing the pouch adjacent to the oral mucosa of a subject, for instance, adjacent to the lip or cheek. In general, saliva can permeate the pouch, and the 6-methylnicotine contained therein can be released from the composition to the saliva of the subject and can pass through the wall of the pouch and across the oral mucosa of the subject, to impart a physiological or psychoactive effect.

The pouch comprises a saliva-permeable non-woven fabric. The pouch can comprise a first saliva-permeable non-woven fabric and a second saliva-permeable non-woven fabric, each comprising an inner surface, wherein the inner surfaces of the first and second saliva-permeable non-woven fabrics are sealed together to form the pouch enclosing the composition. Alternatively, the pouch can be formed from a tube of the saliva-permeable non-woven fabric that is sealed at the open ends. For example, the pouch can be made by a tube filling process well known in the art in which a tube of materials is fed into a filling unit whereby a length of material is cut from the tube and one end is sealed to define the pouch. The pouch is then filled, and the open end is sealed after the pouch is filled. Any known pouch configurations and methods of making pouches can be used. The pouch can be sealed using any known sealing methods so as to minimize or prevent leaking of the composition from the pouch prior to use.

When the pouch is placed adjacent to the oral mucosa, saliva can permeate the non-woven fabric comprising the pouch and carry the 6-methylnicotine, 6-methylnicotine salt, or 6-methylnicotine complex of the composition contained therein to the exterior of the pouch and to the oral cavity of the subject, where the 6-methylnicotine can be orally ingested and/or absorbed through the oral mucosa.

The saliva-permeable non-woven fabric can include, but is not limited to, viscose, rayon, polyurethanes, or a combination thereof. The saliva-permeable non-woven fabric can comprise a heat-sealable non-woven fabric.

The first and second saliva-permeable non-woven fabrics or open ends of a tube of saliva-permeable non-woven fabric can be sealed by any suitable method. The first and second saliva-permeable non-woven fabrics can be heat-sealed.

The saliva-permeable non-woven fabrics can further comprise a binder, such as a thermoplastic binder. The binder can enable the non-woven fabric to be heat-sealed or can improve the heat sealability of the non-woven fabric (as determined, for instance, by an increased seal strength of the heat-sealed non-woven fabric). The binder can include, but is not limited to, one or more vinyl acrylic polymers, one or more acrylic copolymers, or a combination thereof.

Methods of making an oral pouch will be familiar to those of skill in the art.

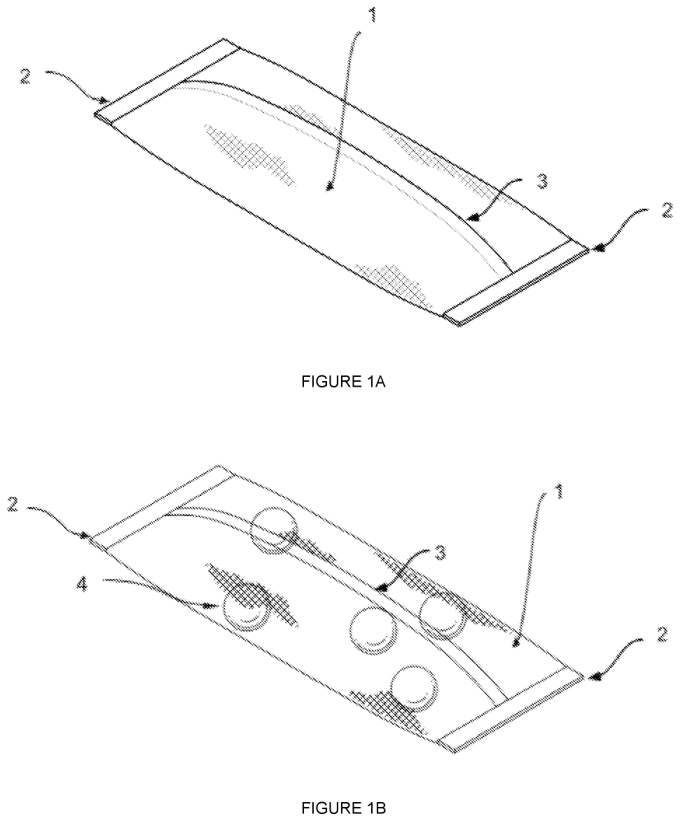

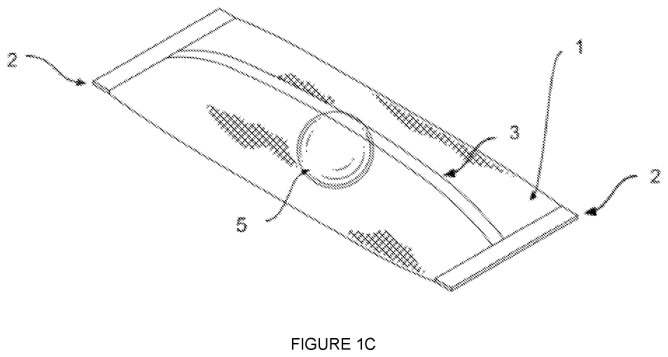

FIGS. 1A to 1C show schematic representations of oral pouches according to the disclosure. FIG. 1A depicts a pouch having a saliva-permeable non-woven fabric 1. The pouch shown in FIG. 1A includes end seals 2 disposed at opposite ends of the pouch and a long seal 3.

FIG. 1B depicts a pouch containing a composition comprising multiple capsules 4;

FIG. 1C depicts a pouch containing a composition comprising a single capsule 5. The composition contained in the pouches depicted in FIGS. 1A to 1C is not explicitly shown.

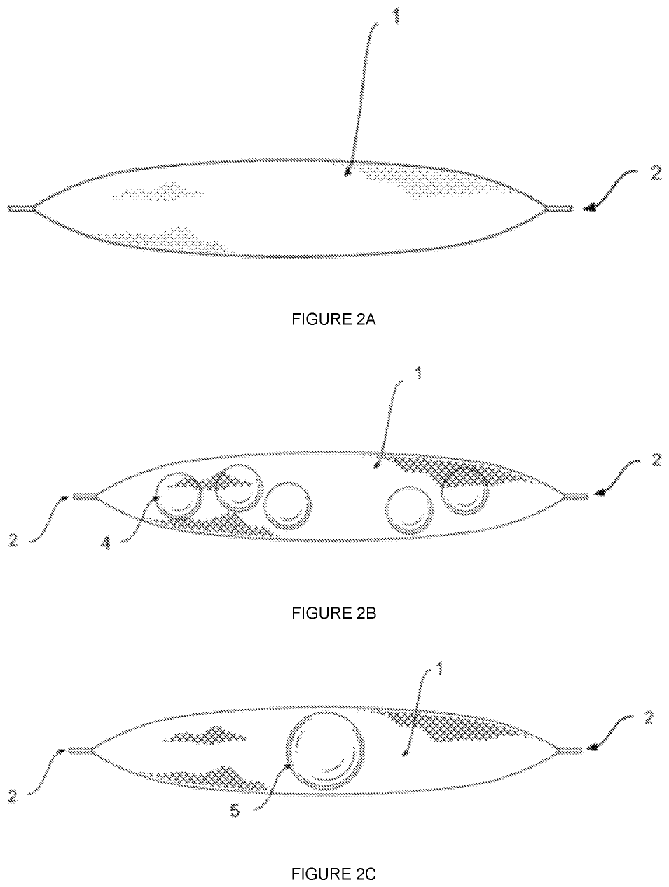

FIGS. 2A-2C show schematic representations of side views of an oral pouch, an oral pouch containing a composition having multiple capsules, and an oral pouch containing a composition having a single capsule, respectively.

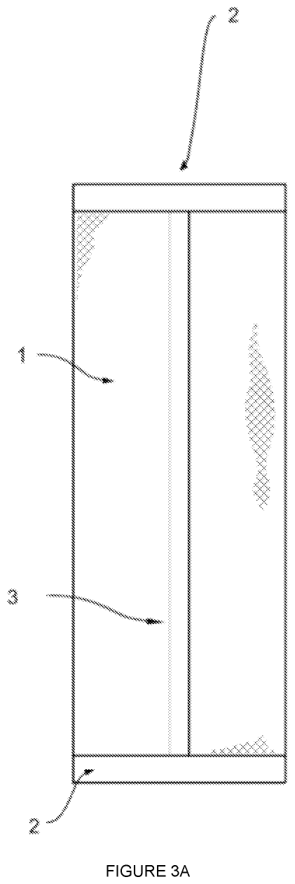

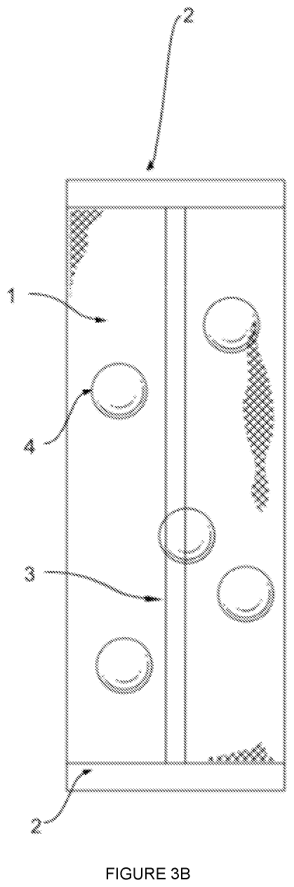

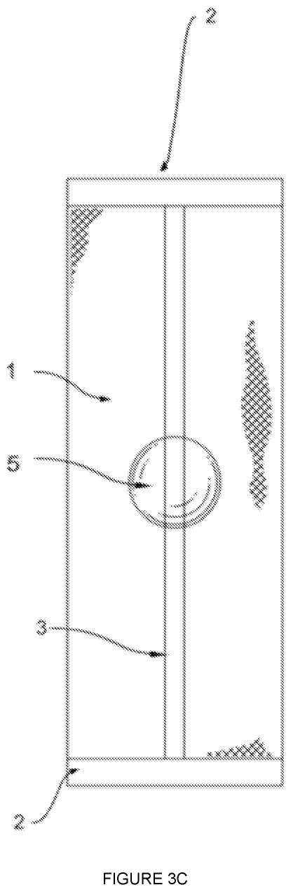

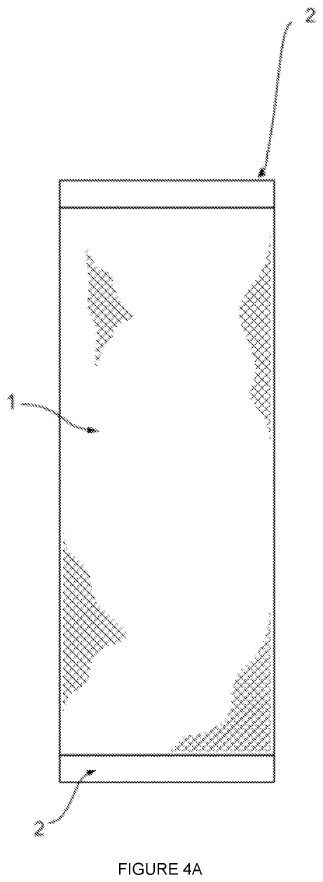

FIGS. 3A-3C show schematic representations of bottom views of an oral pouch, an oral pouch containing a composition having multiple capsules, and an oral pouch containing a composition having a single capsule, respectively.

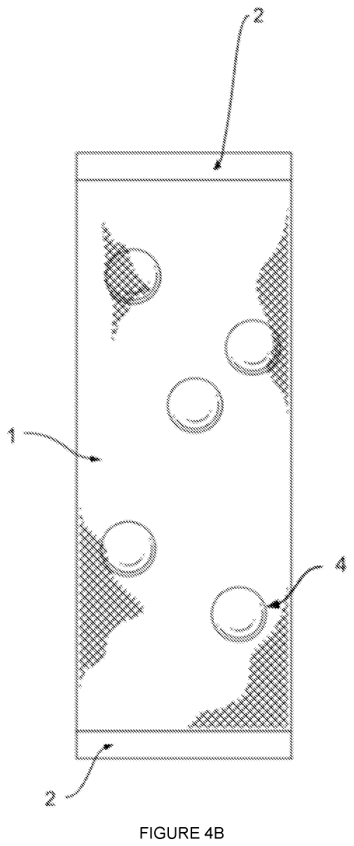

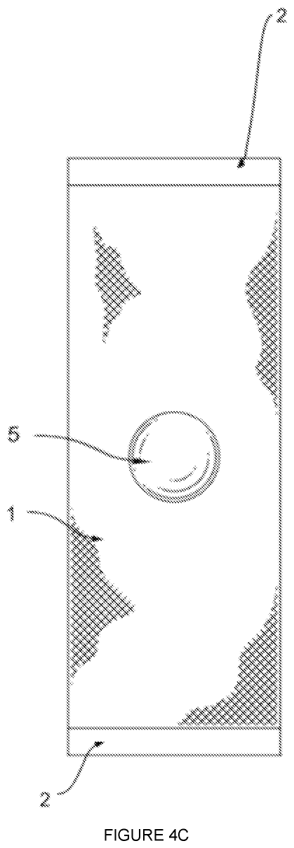

FIGS. 4A-4C show schematic representations of top views of an oral pouch, an oral pouch containing a composition having multiple capsules, and an oral pouch containing a composition having a single capsule, respectively.

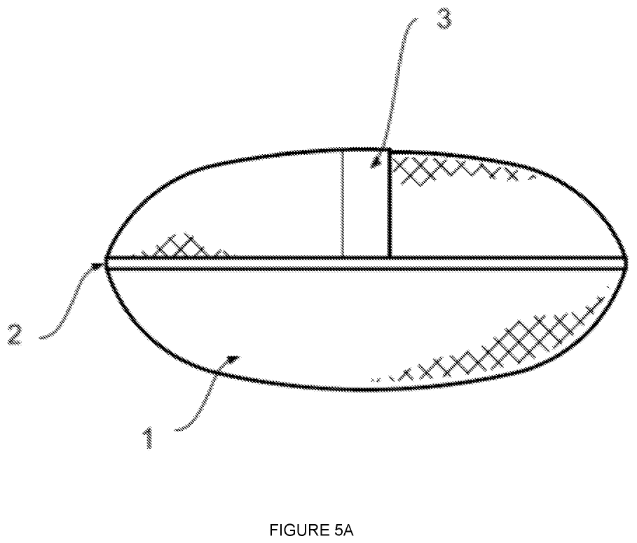

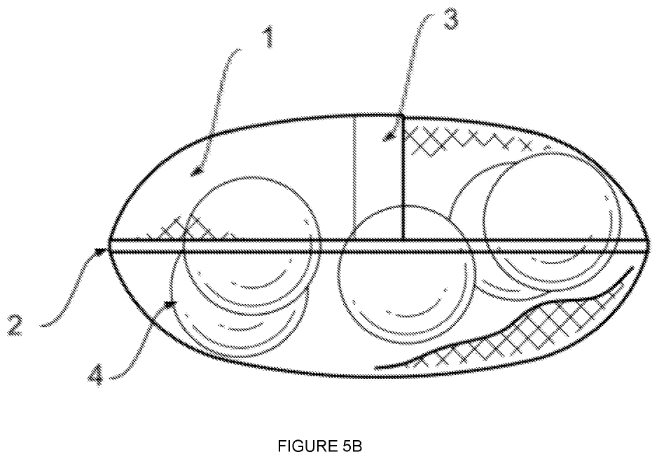

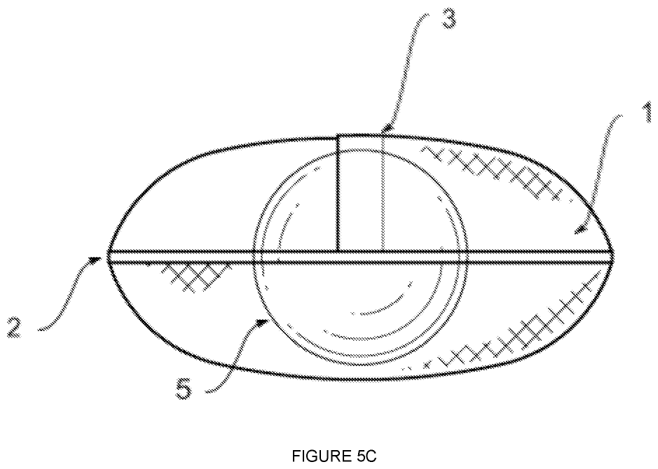

FIGS. 5A-5C show schematic representations of bottom views of an oral pouch, an oral pouch containing a composition having multiple capsules, and an oral pouch containing a composition having a single capsule, respectively.

Though FIGS. 1A-1C, 2A-2C, 3A-3C, 4A-4C, and 5A-5C, depict rectangular pouches, the shape of a pouch according to the disclosure is not particularly limited. The pouch can have any suitable shape including, for example, rectangular, square, circular, and pouches of other shapes. Furthermore, the geometry and alignment of seals of the pouch are not particularly limited. Seals are provided to prevent the composition contained in the pouch from leaking during storage, transport, and final use.

In some aspects, the composition contained in the pouch can contain a 6-methylnicotine salt or a 6-methylnicotine complex. The 6-methylnicotine salt or a 6-methylnicotine complex are selected such that they release 6-methylnicotine upon exposure to saliva as the saliva passes through the pouch. Formulators skilled in the art will be able to identify salts of 6-methylnicotine that are soluble when exposed to saliva and able to release 6-methylnicotine in saliva.

The total amount of 6-methylnicotine in the composition, whether the 6-methylnicotine is present as 6-methylnicotine, a 6-methylnicotine salt, or a 6-methylnicotine complex, can be about 0.1 mg, or about 0.5 mg, 1 mg, 2 mg, 3 mg, 4 mg, 5 mg, 6 mg, 7 mg, 8 mg, 9 mg, or 10 mg, or within a range formed by any such values as endpoints.

### Other Components

The composition can include a filler. The filler can provide bulkiness to the pouch, such that the pouch containing the composition is large enough to be placed in the mouth adjacent to the oral mucosa but is not likely to be swallowed by the user. In general, the filler comprises inert material(s), i.e., materials that are not intended to provide a specific physiological effect. The filler can include, but is not limited to, cellulose, microcrystalline cellulose, maltitol, mannitol, erythritol, allulose, and combinations thereof.

The composition can further include one more additional components including, but not limited to, a binder, a humectant, a flavoring agent, a sweetening agent, and a capsule comprising an inner payload. The additional components can include liquid and/or solid components. The additional components can impart one or more beneficial properties to the pouch, including but not limited to improved flavor, improved product aesthetics, and improved flowability of the composition. The additional components should have low toxicity.

In general, the composition may be administered orally or parenterally. If administered parenterally, the composition may be administered by intravenous injection, subcutaneous injection, local administration, dermal administration, nasal administration, or the like.

In embodiments, the composition can be in the form of a pill, powder, capsule, tablet, granule powder, opercula, orally dissolving granule, sachet, dragee, or powder.

The composition can include a binder. The binder can be added to improve processability of the composition, for instance to improve flowability of the composition in packing machinery used for blending the composition and/or manufacturing the pouch. The binder can include, but is not limited to, modified celluloses, hydroxypropyl cellulose, methyl cellulose, hydroxypropyl methylcellulose (i.e., hypromellose), polyvinyl pyrrolidone, copovidone, gum arabic, pectin, and combinations thereof. When present in the composition, the total amount of binder can be about 1%, about 2%, about 3%, about 4%, about 5%, about 6%, about 7%, about 8%, about 9%, or about 10%, by weight of the composition, or within a range formed by any such values as endpoints.

The composition can include a humectant. The humectant is not particularly limited; propylene glycol, glycerin, and combinations thereof are particularly contemplated.

The composition can include a sweetening agent. The sweetening agent is not particularly limited and can comprise one or more natural sweetening agents, one or more artificial sweetening agents, or a combination thereof. Suitable sweetening agents include, but are not limited to, monosaccharides, disaccharides, trisaccharides, polysaccharides, sugar alcohols, sucrose, glucose, dextrose, maltose, fructose, saccharin, aspartame, acesulfame, neotame sucralose, cyclamates, and combinations thereof.

The composition can include a flavoring agent. The flavoring agent is not particularly limited, and suitable flavoring agents will be familiar to those of skill in the art. Flavoring agents can include apple, cinnamon, orange, mango, citrus, lemon, peppermint, mint, menthol, wintergreen, tobacco, coffee, vanilla, lime, and peach flavoring agents, and combinations thereof. The flavoring agent can be a liquid or a solid.

The composition can include a pH adjuster. The pH adjuster is not particularly limited and can include, but is not limited to, carbonates, bicarbonates, sesquicarbonates, acetates, glycinates, gluconates, borates, glycerophosphates, citrates, phosphates (e.g., mono-, di-, or trihydrogenphosphates), hydroxides (e.g., sodium hydroxide or potassium hydroxide), and combinations thereof. Sodium carbonate, sodium bicarbonate, and combinations thereof are particularly contemplated. When present, the pH adjuster can be present in the composition in an amount such that the pH of the composition, determined as the pH of a solution of the composition in deionized water, is in a range of 7.0 to 9.5. Without intending to be bound by theory, it is believed that when the pH of the composition is in a range of 7.0 to 9.5, 6-methylnicotine dissolved and/or dispersed in saliva in the oral cavity of a subject using the pouch is better able to be absorbed by and pass through the oral mucosa.

In embodiments, the composition is free of a pH adjuster. In particular, compositions comprising a 6-methylnicotine salt or a complex of 6-methylnicotine with a cyclodextrin or ion exchange resin can be free of a pH adjuster and can have a pH in a range of 7.0 to 9.5.

The composition can contain a capsule containing an inner payload containing an additive. The capsule can comprise a capsule wall encapsulating a payload that comprises one or more liquids, one or more solids, or a or a mixture of one or more liquids and one or more solids. Components of the payload can include, but are not limited to, flavoring agents, sweeteners, MCT oil, propylene glycol, glycerol, safflower oil, and combinations thereof. In general, the capsule, when present in the composition, does not contain the 6-methylnicotine of the composition.

The total amount of composition in a pouch can be about 200 mg, or about 300 mg, or about 400 mg, or about 500 mg, or about 600 mg, or about 700 mg, or about 800 mg, or within a range formed by any such values as endpoints. For example, the amount of composition in a pouch can be in a range of about 300 mg to about 600 mg.

In embodiments, the composition comprises a blend of dry ingredients and optionally one or more liquid ingredients. The composition can comprise one or more powdered solids, one or more granulated solids, or a combination thereof. The composition can be dry or substantially dry. For instance, the composition can have a moisture content of less than about 15%, less than about 10%, or less than about 5%, or less than about 3%, or less than about 1%, by weight of the composition.

Table 1 lists typical components of the composition according to the disclosure. Such composition can be suitable, for example, for inclusion in an oral pouch. The listed components and amounts of each component and total composition are included for example purposes only and are not intended to be limiting.

TABLE 1

| | | Component |
|---|---|---|
| Component | Example(s) | Weight |
| Active | 6-methylnicotine salt; | ~5-100 mg |
| ingredient | 6-methylnicotine β-cyclodextrin | |
| | complex; 6-methylnicotine polacrilex | |
| Filler | Microcrystalline cellulose, maltitol, | ~50-300 mg |
| | mannitol, erythritol, allulose | |
| Sweetener | Acesulfame potassium, sucralose, | ~1-10 mg |
| | saccharin, stevia | |
| Flavoring | Flavoring agent or blend of | ~5-35 mg |
| | flavoring agents | |
| pH Adjuster | Sodium carbonate, sodium bicarbonate | ~0-25 mg |
| Humectant | Propylene glycol, glycerin | ~10-100 mg |
| Binder | Hydroxypropyl cellulose, polyvinyl | ~3-30 mg |
| | pyrrolidone, copovidone, methyl | |
| | cellulose, hypromellose | |
| | Composition Weight | ~300-600 mg |

### Other Product Forms

### Oral Dissolvable Film

In other aspects, the smoking cessation aid comprises an oral dissolvable film, or strip, comprising 6-methylnicotine and a film-forming saliva-soluble polymer. The oral dissolvable film can be placed in a subject's oral cavity, for instance against the cheek, and the 6-methylnicotine can be absorbed by the oral mucosa, aided by the subject's saliva. Without intending to be bound by theory, it is believed that absorption of 6-methylnicotine through the oral mucosa can generally be achieved more quickly with an oral dissolvable film compared to a pouch.

The film can comprise 6-methylnicotine, a 6-methylnicotine salt, or a 6-methylnicotine complex. The amount of 6-methylnicotine in the film can be in a range of 0.5% to 10% by weight of the film.

The film comprises a film-forming saliva-soluble polymer. Suitable film-forming saliva-soluble polymers for an oral dissolvable film of the disclosure include, but are not limited to, pullulan, starch and modified starches, sodium alginate, pectin, maida, cellulose and modified celluloses, hydroxypropyl cellulose, hypromellose, and combinations thereof. Film-forming polymers for an oral dissolvable strip will be familiar to those of skill in the art. The film-forming saliva-soluble polymer can be present in the film in an amount of about 35%, or about 40%, 45%, 50%, 55%, 60%, 65%, 70%, 75%, 80%, 85%, 90%, or 95%, by weight of the film, or within a range formed by any such values as endpoints.

The film can further comprise one or more additional components, including but not limited to a plasticizer, a stabilizer, a sweetener, a pH adjuster, a flavoring agent, a coloring agent, and combinations thereof.

In general, a plasticizer is a liquid or solid that can be added to a material (for instance, a resin or polymer) to make the material more flexible and easier to process, for instance, by lowering the material's glass transition temperature. Polymer films containing a low level of plasticizer may become brittle, difficult to process, or prone to breaking. Polymer films containing excessive plasticizer may be soft, weak, or difficult to process for a desired use. Though water is generally recognized as an efficient plasticizer for some polymers, its utility as a plasticizer can be limited due to its volatility. The plasticizer for an oral dissolvable film of the disclosure can include, but is not limited to, polyols, glycerin, propylene glycol, polyethylene glycol, and combinations thereof. The plasticizer can be present in an amount of up to about 20%, or up to about 15%, or up to about 10%, or up to about 5%, by weight of the film.

The film can comprise a stabilizer, such as a surfactant, to aid in maintaining homogeneity of the film and of any formulations that are made in the course of producing the film. Though the film is intended to be water- and saliva-soluble, the film can include components with low or no water solubility (many flavoring agents, for instance, have low to no water solubility), and the addition of a surfactant can improve miscibility of all film components. The stabilizer is not particularly limited; Polysorbate 20 and Polysorbate 80 are particularly contemplated. The stabilizer can be present in an amount of up to about 10%, or up to about 9%, 8%, 7%, 6%, or 5%, by weight of the film.

The film can comprise a sweetening agent. The sweetening agent is not particularly limited and can comprise one or more natural sweetening agents, one or more artificial sweetening agents, or a combination thereof. Suitable sweetening agents include, but are not limited to, stevia, xylitol, acesulfame potassium, sucralose, saccharin, aspartame, neotame, and combinations thereof. The sweetening agent can be present in the film in an amount up to about 5%, or up to about 4%, 3%, 2%, or 1%, by weight of the film.

Table 2 lists typical components of the composition of an oral dissolvable film according to the disclosure. The listed components and amounts of each component are included for example purposes only and are not intended to be limiting.

TABLE 2

| | | Weight % |
|---|---|---|
| Component | Example(s) | of strip |
| Active | 6-methyl nicotine, 6-methylnicotine salt, | 0.1-10% |
| ingredient | 6-methylnicotine cyclodextrin complex, | |
| | 6-methylnicotine polacrilex | |
| Film former | Pullulan, sodium alginate, pectin, maida, | 35-55% |
| | hydroxypropyl cellulose, carrageenan, | |
| | starch | |
| Plasticizer | Glycerol, propylene glycol, polyethylene | 0-30% |
| | glycol | |
| Stabilizer | Polysorbate 20, Polysorbate 80 | 0-10% |
| Sweetener | Stevia, acesulfame potassium, sucralose, | 0-25% |
| | saccharin, aspartame, neotame | |
| pH Adjuster | Citric acid, malic acid, lactic acid, | 0-10% |
| | salicylic acid | |
| Flavoring agent | Flavor Blend | q.s. |
| Coloring agent | Red 40 Lake, Blue 1 Lake, Yellow 5 Lake | q.s. |

### Chewing Gum

In another aspect, the smoking cessation product comprises a chewing gum comprising 6-methylnicotine. The gum can include 6-methylnicotine in its racemic form, purified to (S)-6-methylnicotine, or purified to (R)-6-methylnicotine, combined with a gum base mixture. The 6-methylnicotine can be released from the gum upon mastication of the gum, wherein the 6-methylnicotine can be absorbed through the oral mucosa. Further release of the active ingredient can come from multiple iterations of chewing the gum to release the 6-methylnicotine and allowing the released ingredient to be absorbed when placed into the gumline or “parked.”

The chewing gum can contain a 6-methylnicotine salt or a 6-methylnicotine complex with a cyclodextrin or with an ion exchange resin. In one embodiment, 6-methylnicotine is complexed with an ion exchange resin to control the release of the 6-methylnicotine. Complexing the 6-methylnicotine can also improve the stability (for instance, with respect to degradation and oxidation) and shelf life of the chewing gum; the gum may be prone to degradation if 6-methylnicotine is used in its free (base) form. Those skilled in the art will be able to identify appropriate additions to form a salt or additional complexes so that the 6-methylnicotine maintains acceptable stability and can be charged into the gum base as a solid powder.

A pH adjusting agent can optionally be employed such that the gum has a pH of 7-9.5, determined as the pH when the gum formulation is exposed to purified water. Suitable pH adjusting agents include, but are not limited to, carbonates, including mono-, bi-, and sesquicarbonates; acetates; glycinates; gluconates; borates; glycerophosphates or citrates of alkaline metals or ammonium; phosphates including mono-, di-, and trihydrogenphosphates; metal hydroxides such as sodium hydroxide and potassium hydroxide; and mixtures thereof.

Additional components combined with the 6-methylnicotine complex or salt in the gum base can include one or more fillers, sweeteners, flavoring, and plasticizers, or a combination thereof. In some embodiments a hard shell coating can be applied to the chewing gum which can improve consumer perception and the stability of the gum. A hard shell can be formed, for instance, by coating the gum with a binder and applying syrups, flavoring, sweeteners, coloring agents, polishing agents, or a combination thereof.

The gum can contain one or more plasticizers. Plasticizers can help soften the gum base so that additional ingredients can be homogeneously mixed throughout the gum base, allowing consumers to more easily masticate the gum with their teeth to release the active ingredient. Suitable plasticizers can include, but are not limited to, glycerin, triacetin, propylene glycol, polyethylene glycols (PEGs), and MCT oil.

In embodiments, a hard shell coating can be applied to the chewing gum. The hard coating can improve consumer perception and stability of the gum. A hard shell can be formed, for instance, by coating a core comprising the gum base with a binder. Suitable binder materials include, but are not limited to, gum arabic, carrageenan, pectin, and other polysaccharides. When the core is coated with binder, syrups comprising maltitol and water can be added to sweeten the gum coating. Additional sweeteners such as sugar, acesulfame potassium, saccharin, neotame, sucralose, stevia, allulose, and additional identified sweetening ingredients suitable for coating can also be added to the coating to improve the taste profile of the chewing gum. Flavoring, as a liquid and/or as a spray dried solid, can also be added to the coating. Depending on the preference of the consumer, coloring agents such as titanium dioxide or other whitening or coloring agents known to those skilled in the art can be added to improve appearance. In a final step, the coating can be polished with shellac or camauba wax to provide a shiny and smooth appearance.

Table 3 lists typical components of the composition of a chewing gum according to the disclosure. The listed components and amounts of each component are included for example purposes only and are not intended to be limiting.

TABLE 3

| | | Weight % |
|---|---|---|
| Component | Example(s) | of gum |
| Active | 6-methylnicotine polacrilex, | 0.5-5% |
| ingredient | 6-methtylnicotine bitartrate, | |
| | 6-methylnicotine β-cyclodextrin | |
| Gum base | Gum Base | 35-55% |
| Filler | Calcium carbonate, sorbitol, maltitol, talc | 0-20% |
| Plasticizer | Glycerol, propylene glycol, polyethylene | 0-10% |
| | glycol, MCT oil, safflower oil, acetylated | |
| | monoglycerides | |
| Sweetener | Stevia, acesulfame potassium, sucralose, | 0-5% |
| | allulose, saccharin, aspartame, neotame | |
| pH Adjuster | Sodium bicarbonate, sodium carbonate | 0-5% |
| Flavor | Flavor blend | q.s. |
| Coloring Agent | Red 40 Lake, Blue 1 Lake, Yellow 5 Lake | q.s. |
| Binder | Gum Arabic, carrageenan, pectin, | q.s. |
| (coating) | hydroxypropyl cellulose, polyvinyl | |
| | pyrrolidone, copovidone, methyl cellulose, | |
| | hypromellose | |
| Sweetener | Maltitol syrup, acesulfame potassium, | q.s. |
| (coating) | saccharin, aspartame, sucralose | |
| Coloring agent | Titanium Dioxide | q.s. |
| (coating) | | |
| Polishing agent | Carnauba wax | q.s. |
| (coating) | | |
| | Total weight | 1.0-1.5 g |

### Oral or Nasal Spray

In another aspect, the smoking cessation product comprises a spray, such as an oral spray or a nasal spray, comprising 6-methylnicotine. The spray can comprise 6-methylnicotine in its racemic form, purified to(S)-6-methylnicotine, or purified to (R)-6-methylnicotine, added to a liquid carrier to form a liquid spray. In embodiments, the carrier comprises phosphate buffered saline. Buffered saline improves mucociliary clearance and provides pH stability. Optionally, the spray can comprise one or more pH adjusting agents to maintain a pH of the spray of 7-9.5.

6-methylnicotine can be present in the spray in its free form, as a water-soluble 6-methylnicotine salt, or a combination thereof. 6-methylnicotine can be added as either a water soluble salt or in its free form. Suitable 6-methylnicotine salts include, but are not limited to, salts of 6-methylnicotine with benzoic acid, malic acid, salicylic acid, citric acid, levulinic acid, or tartaric acid.

In an embodiment of the spray formulation, 6-methylnicotine is first added in its free (base) form, followed by addition of an acid to decrease the pH to be in a range of 7.0 to 9.5. In this embodiment, some of the 6-methylnicotine may remain in its un-ionized (free) form.

As will be known to those skilled in the art, a variety of agents may be used to produce a desired viscosity of the spray, including cellulose, substituted celluloses such as carboxymethyl cellulose and methyl cellulose, gum arabic and polyethylene glycol. The desired viscosity may also be produced by use of an oil emulsion, the oil phase being any suitable nasally acceptable oil including, for example, lanolin or beeswax. Any viscosity producing agent used must, of course, be pharmaceutically acceptable and well tolerated by the nasal mucosa.

The 6-methylnicotine-containing composition of the invention may be applied to the nose or oral cavity by any suitable atomiser or spray device which produces a spray of droplet size greater than about 10 microns. For example, conventional venturi-type atomisers such as are used for nasal decongestants or metered dose spray devices such as are used for nasal steroid application may be employed. These devices produce 98% of droplets greater than 16 microns and a majority of droplets of approximately 100 to 200 microns. As will be understood by those skilled in the art, the viscosity of the composition of the invention should be optimized for the type of spray device employed.

When a nasal spray of the disclosure is used, 6-methylnicotine is not drawn into the user's airways beyond the nose or oral cavity, thus avoiding respiratory irritation and allowing the use of higher 6-methylnicotine concentrations.

A preservative can be included in the spray, to reduce or prevent microbial growth in the spray and increase shelf life of the spray. The preservative can be present in the spray in an amount sufficient to inhibit or prevent microbial growth (e.g., growth of aerobic and/or anaerobic bacteria). For example, the preservative can be present in an amount of up to about 0.5%, or up to about 1.0%, or up to about 2.0%, by weight of the spray. The preservative can include, but is not limited to, benzyl alcohol, phenethyl alcohol, or a mixture thereof.

When administered directly, 6-methylnicotine may be unpalatable to some consumers, introducing the risk of the consumer refusing to use the smoking cessation product and returning to tobacco use as a result. In order to increase the palatability of the oral or nasal spray, flavoring, cooling agents, and natural analgesics may be used.

Optionally, the spray can include one or more surfactants to improve formulation stability.

Natural analgesics suitable for soothing the application of 6-methylnicotine include aloe, linalool, kava, methyl salicylate, menthol, eugenol, thymol, eunal, propinal, acyclic monoterpenoids, monocyclic monoterpenoids, bicyclic monoterpenoids, and sesquiterpenoids. In addition, various alkaloids, including harmala alkaloids, harman, norharman, hermol, harmine, harmalol, harmaline, tetrahydroharman, tetrahydroharmane may be employed to similar effect. Identified flavonoids that can be employed in the product include flavone chemicals such as chrysin, apigenin, 6-methylapigenin, baicalein, baicalin, scutellarein, hispidulin, cirsilineol, wogonin, flavone glycosides such as isoorientin, orientin, isovitexin, and vitexin. The flavonol kaemferol, flavanone glabrol, flavanol (−)-epigallocatechin-3-gallate, and the chalcone isoliquiritigenin may also be used as natural analgesics to soothe unpleasant sensations from the oral and nasal spray.

Table 4 lists typical components of the composition of a spray according to the disclosure. The listed components and amounts of each component are included for example purposes only and are not intended to be limiting.

TABLE 4

| | | Weight % |
|---|---|---|
| Component | Example(s) | of spray |
| Active ingredient | 6-methylnicotine | 0.5-5% |
| Carrier | Phosphate buffered saline | 90-99% |
| pH Adjuster | Citric acid, levulinic acid, malic acid, | 0.1-1% |
| | salicylic acid | |
| Aroma | Methyl salicylate, menthol, L-carvone | 0-1% |
| Analgesic | Aloe, linalool, kava, camphor | 0-4% |
| Surfactant | TWEEN 20, TWEEN 80 | 1-4% |
| Preservative Agent | Benzyl alcohol | 0.1-0.6% |
| Preservative Agent | Phenethyl alcohol | 0.1-0.35% |
| | Total weight | 1.0-1.5 g |

### Methods of Treatment

Methods of the disclosure include using a composition of the disclosure to deliver an effective amount of 6-methylnicotine to a subject in need thereof, such as for treatment of nicotine addiction and/or to alleviate (reduce or eliminate) nicotine withdrawal symptoms and/or simulate a nicotine physiological effect. Treating nicotine addiction can include, for instance, one or more of reducing consumption of nicotine (such as by consuming an effective alternative to nicotine), reducing symptoms of nicotine withdrawal, and reducing tobacco usage.

Compositions according to the disclosure can be used in substantially the same manner as other smoking cessation products, such as nicotine-containing smoking cessation products. For instance, an oral pouch according to the disclosure can be placed against the cheek or lip of a subject, where saliva can permeate the pouch, release the 6-methylnicotine contained therein, and deliver the 6-methylnicotine to the oral mucosa where it can be absorbed.

As demonstrated in the examples that follow, using a smoking cessation product according to the disclosure can provide beneficial effects, including reduction of nicotine cravings and satisfying physiological effects, without consuming nicotine. Furthermore, as demonstrated in the examples, beneficial effects resulting from using a smoking cessation product according to the disclosure could be achieved using a lower dose of active ingredient compared to the dose of nicotine in a similar nicotine-containing smoking cessation product. The dosage of 6-methylnicotine for smoking cessation products of the disclosure can be from 0.5 mg to 6.0 mg, with variations depending on product type and desired level of physiological effects. Lower doses of 6-methylnicotine may still provide beneficial physiological effects, while higher doses may be useful for subjects experiencing severe nicotine withdrawal symptoms.

Without intending to be bound by theory, it is believed that 6-methylnicotine can bind to nicotinic receptors, in part due to its chemical and/or structural similarity to nicotine, and can accordingly satisfy cravings associated with nicotine use. It is further believed that 6-methylnicotine binds strongly to nicotinic receptors and that ingestion of 6-methylnicotine can produce a similar or stronger physiological response compared to ingestion of the same dose amount of nicotine.

Achieving similar or stronger physiological response with a lower dose of active ingredient (i.e., 6-methylnicotine) can also provide a long-term benefit of enabling lower cumulative alkaloid consumption over the course of product use. Furthermore, without intending to be bound by theory, it is believed that 6-methylnicotine is less addictive than nicotine, and that treating nicotine addiction according to methods of the disclosure carries less risk of addiction or relapse to tobacco use compared to nicotine replacement therapy methods involving nicotine consumption.

### EXAMPLES

The following examples are provided for illustration and are not intended to limit the scope of the invention.

In general, the following examples compared the effects of 6-methylnicotine consumption to nicotine consumption.

### Example 1: Oral Pouches

Six volunteer subjects who are regular users of nicotine pouches containing 8 mg of nicotine were each administered an oral pouch of the disclosure containing 3 mg of 6-methylnicotine. Each subject used the 6-methylnicotine pouch in the same manner as a nicotine pouch, by placing the pouch inside the mouth and adjacent to the lip and gum. All subjects reported that using the pouch of the disclosure containing 3 mg of 6-methylnicotine provided a more potent physiological effect, such as a “head rush” experienced during pouch usage, compared to using a pouch containing 8 mg of nicotine. The subjects also reported zero desire to use nicotine- or tobacco-based products after use of the pouch of the disclosure.

Example 1 demonstrates that a 6-methylnicotine pouch according to the disclosure provided physiological effects more potent than effects provided by a nicotine pouch, and that the similar or potent physiological effects provided by a pouch according to the disclosure were achieved using a much lower dosage of the active ingredient compared to the dosage of the nicotine pouch (3 mg vs. 8 mg). This example further demonstrates that the use of 6-methylnicotine, as delivered by a smoking cessation product according to the disclosure, alleviated cravings associated with nicotine addiction.

### Example 2: Oral Dissolvable Films

Four volunteer subjects were provided with an oral dissolvable film according to the disclosure containing 1.6 mg of 6-methylnicotine and an oral dissolvable film containing 2 mg of nicotine. Each subject placed the nicotine strip against the inside of their cheek and noted their physiological response. Subjects abstained from nicotine or tobacco usage for 1 hour after consuming the nicotine strip, then placed the 6-methylnicotine strip according to the disclosure against the inside of their cheek and noted their physiological response. All subjects reported a stronger physiological response (as a perceived “head rush”) from consuming the 6-methylnicotine strip compared to consuming the nicotine strip.

Example 2 demonstrates that 6-methylnicotine provided physiological effects more potent than those provide by nicotine, even when using a lower dosage of 6-methylnicotine compared to nicotine (1.6 mg vs. 2 mg).

### Example 3: Blood Pressure Effects

Five volunteer subjects compared the effects of nicotine consumption and 6-methylnicotine consumption on blood pressure Subjects were blindfolded and had their blood pressure measured before using a pouch (to provide a baseline blood pressure) and re-measured approximately 1 minute after placing a nicotine pouch or a 6-methylnicotine pouch adjacent to the gum line. All subjects performed the test with a nicotine pouch and with a 6-methylnicotine pouch on the same day with a one-hour break between the tests. No significant difference between blood pressure following usage of a nicotine pouch and blood pressure following usage of a 6-methylnicotine pouch was observed for any of the subjects.

The foregoing description is given for clearness of understanding only, and no unnecessary limitations should be understood therefrom, as modifications within the scope of the invention may be apparent to those having ordinary skill in the art.

All patents, publications and references cited herein are hereby fully incorporated by reference. In case of conflict between the present disclosure and incorporated patents, publications and references, the present disclosure should control.

1. A smoking cessation product for oral delivery, comprising:
a. an oral pouch comprising a saliva-permeable non-woven fabric; and
b. a composition contained within the oral pouch, the composition comprising:
i. a 6-methylnicotine salt or complex; and
ii. a filler.

2. The smoking cessation product of claim 1, wherein the composition further comprises one or more of a humectant, a flavoring agent, a sweetening agent, and a capsule comprising an inner payload.

3. The smoking cessation product of claim 1, wherein the 6-methylnicotine salt is selected from the group consisting of 6-methylnicotine hydrochloride, 6-methylnicotine dihydrochloride, 6-methylnicotine monotartrate, 6-methylnicotine bitartrate, 6-methylnicotine bitartrate dihydrate, 6-methylnicotine sulphate, 6-methylnicotine malate, 6-methylnicotine benzoate, 6-methylnicotine citrate, 6-methylnicotine levulinate, 6-methylnicotine zinc chloride monohydrate, 6-methylnicotine salicylate, and combinations thereof.

4. The smoking cessation product of claim 1, wherein the 6-methylnicotine complex is selected from the group consisting of 6-methylnicotine cyclodextrin complex, 6-methylnicotine polacrilex, and combinations thereof.

5. The smoking cessation product of claim 4, wherein the 6-methylnicotine cyclodextrin complex comprises β-cyclodextrin (beta-cyclodextrin).

6. The smoking cessation product of claim 1, wherein the filler is selected from the group consisting of microcrystalline cellulose, maltitol, mannitol, erythritol, allulose, and combinations thereof.

7. The smoking cessation product of claim 1, wherein the composition further comprises a binder selected from the group consisting of hydroxypropyl cellulose, polyvinyl pyrrolidone, copovidone, methyl cellulose, hypromellose, gum arabic, pectin, and combinations thereof.

8. The smoking cessation product of claim 1, wherein the composition further comprises a pH adjuster comprising sodium carbonate, sodium bicarbonate, or a combination thereof.

9. The smoking cessation product of claim 8, wherein the pH of an aqueous solution of the composition is in a range of 7.0 to 9.5.

10. The smoking cessation product of claim 1, wherein the composition is free of a pH adjuster.

11. The smoking cessation product of claim 2, wherein the humectant comprises glycerin, propylene glycol, or a combination thereof.

12. The smoking cessation product of claim 2, wherein the sweetening agent is selected from the group consisting of monosaccharides, disaccharides, trisaccharides, polysaccharides, sugar alcohols, sucrose, glucose, dextrose, maltose, fructose, saccharin, aspartame, acesulfame, neotame sucralose, cyclamates, and combinations thereof.

13. (canceled)

14. The smoking cessation product of claim 1, wherein the saliva-permeable nonwoven fabric comprises viscose, rayon, a thermoplastic polyurethane, or a combination thereof.

15. (canceled)

16. The smoking cessation product of claim 1, wherein the oral pouch comprises a first saliva-permeable non-woven fabric and a second saliva-permeable non-woven fabric, each comprising an inner surface,
wherein the inner surfaces of the first and second saliva-permeable non-woven fabric are sealed together to form the pouch.

17.-18. (canceled)

19. The smoking cessation product of claim 1, wherein the composition comprises 0.5 mg to 10 mg of the 6-methylnicotine salt or complex.

20.-21. (canceled)

22. The smoking cessation product of claim 1, wherein the 6-methylnicotine comprising the 6-methylnicotine salt or complex comprises at least 90% (S)-6-methylnicotine, based on the total weight of 6-methylnicotine.

23.-24. (canceled)

25. A method of treating a subject for addiction to nicotine, comprising:
orally administering to a subject in need thereof the smoking cessation product of claim 1, wherein the smoking cessation product is disposed against the oral mucosa of the subject, wherein upon contact with the oral mucosa, saliva permeates the smoking cessation product and dissolves the composition for absorption of the 6-methylnicotine through the oral mucosa.

26. A method of reducing nicotine withdrawal symptoms, comprising:
orally administering to a subject in need thereof the smoking cessation product of claim 1, wherein the smoking cessation product is disposed against the oral mucosa of the subject, wherein upon contact with the oral mucosa, saliva permeates the smoking cessation product and dissolves the composition for absorption of the 6-methylnicotine through the oral mucosa.

27. A method of reducing nicotine usage, comprising:
orally administering to a subject in need thereof the smoking cessation product of claim 1, wherein the smoking cessation product is disposed against the oral mucosa of the subject, wherein upon contact with the oral mucosa, saliva permeates the smoking cessation product and dissolves the composition for absorption of the 6-methylnicotine through the oral mucosa.

28. An oral dissolvable film for delivering 6-methylnicotine to the oral mucosa, comprising:
a. a film-forming saliva-soluble polymer; and
b. 6-methylnicotine or a 6-methylnicotine salt.

29.-50. (canceled)
